(19) United States
(12) Patent Application Publication
(10) Pub. No.: US 2012/0267275 A1
(43) Pub. Date: Oct. 25, 2012

(54) Cases for concealing alternative nutrition and/or intravenous bags hanging from an IV stand pole

(76) Inventor: Dovie D. Gilmore, Milwaukee, WI (US)

(21) Appl. No.: 13/066,691

(22) Filed: Apr. 22, 2011

(51) Int. Cl. A61B 19/02

(52) U.S. Cl. 206/438

(57) ABSTRACT

The present is an alternative nutrition and/or intravenous bag concealing sack fabricated from a series of fabric, plastic and/or paper components and an elastic band, positioned in the bottom seam of the sack, providing a means which permit the sack free movement horizontally in and out covering adjacent surfaces.

I claim priority to provisional application No. 61/342,673 “Cloth sewn cases of concealing alternative nutrition and/or intravenous bags hanging from a stand pole”, filed Apr. 19, 2010.

### BACKGROUND OF THE INVENTION

1.) The field of the present invention relates to a firm fitted form of a pillowcase for use of concealing alternative nutrition and/or intravenous bags. The concept of a soft fabric, plastic or paper cover for alternative nutrition and/or intravenous as to provide privacy for individual recipients.

2.) The prior art conventional pillowcases for pillows, such as U.S. Pat. No. 5,084,928 are described as formed of one or more sheets of soft, flexible fabric such as cotton either woven in a tubular shape or as a flat sheet of material.

### SUMMARY OF THE INVENTION

The alternative nutrition and/or intravenous bag concealing sack provides a fabric, plastic and/or paper provision of a pillowcase designed to facilitate disclosure of alternative nutrition and/or intravenous bags hanging from an intravenous stand pole. The concealing sacks vertical movement and the free horizontal movement of the elastic band sewn into the bottom of the sack provides disclosure of multiple adjacent surfaces.

### DESCRIPTION OF THE DRAWINGS

FIG. 1 is a perspective view of the concealing sack 200 as seen in FIGS. 4, 6 and covering intravenous bag 300 and pole 100.

FIG. 2 is a plan view of the sack fabric pattern pieces 110 and 120 before they are stitched and the elastic band 160.

FIG. 3a is a view of the sack pattern pieces front and back sewn or glued together 130.

FIG. 3b is a perspective view of the elastic band 160 as seen in FIG. 2 that folds upon itself and stitched at each end 160a and 160b.

FIG. 4 is a front view 200 corresponding with FIG. 1 shows elastic band 160 stitched 250 and 260 to bottom seam of sack horizontally.

FIG. 5 is a bottom end elevated view of sack showing empty cavity 230 stitches 260 and elastic band 160.

FIG. 6 is a side view showing seam 240 corresponding with FIG. 4.

### DETAILED DESCRIPTION OF THE INVENTION

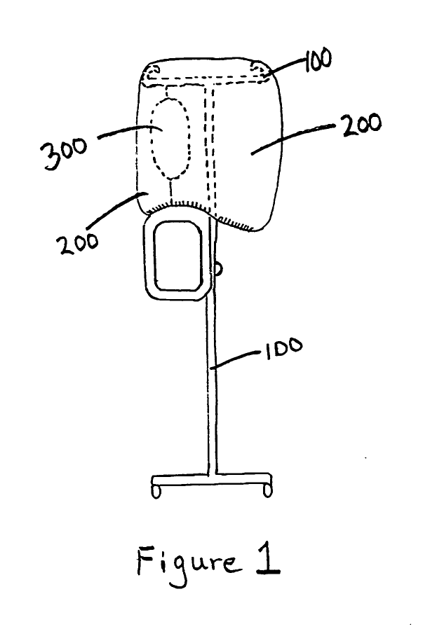

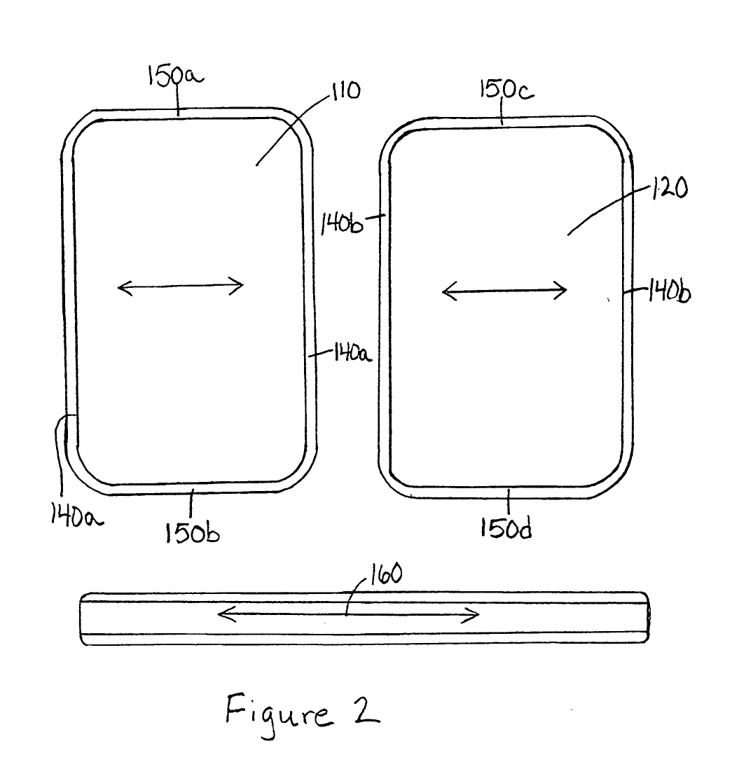

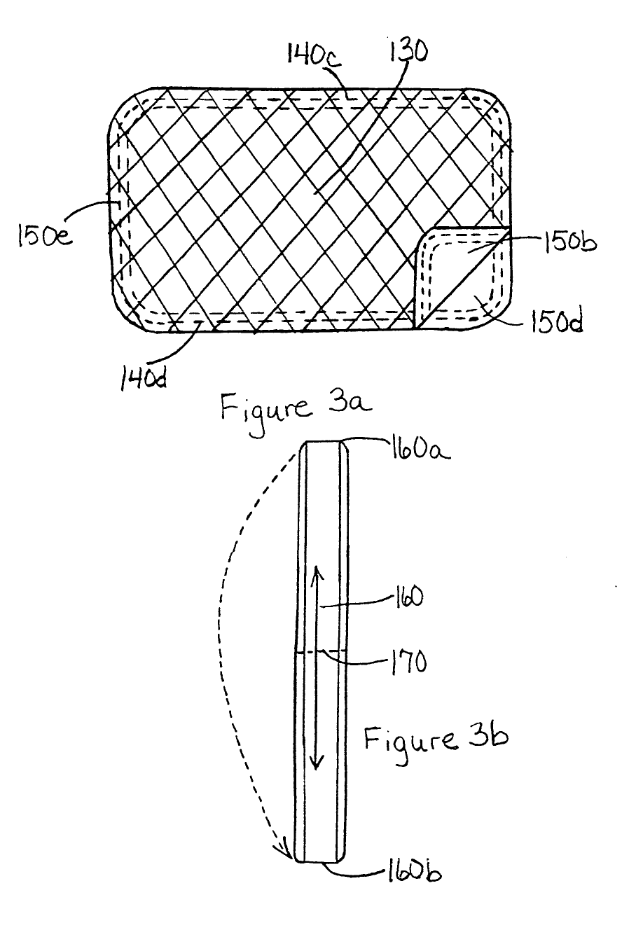

Referring to drawings FIGS. 2, 3a, 3b, 4, 5, 6 and firstly FIG. 1, shows an exemplary embodiment sack 200 formed of cloth fabric, plastic or paper concealing the top portion of an intravenous pole 100 and bag 300 hanging from the pole. The peripheral extremities of the rectangular fabric pattern pieces 110 and 120 long sides 140a and 140b form one set of adjacent sides 140c and 140d as do the top short sides 150a and 150c form 150e as seen in FIG. 3a. Each of the long sides 140c and 140d are between about 28 to 40 inches in length and each of the short sides top and bottom 150b, 150d, and 150e are between about 20 to 24 inches in length.

Each of the long sides pattern pieces 140a and 140b is permanently connected together forming seam 240 seen in FIG. 6 as do short sides pattern pieces 150a and 150c is connected together permanently.

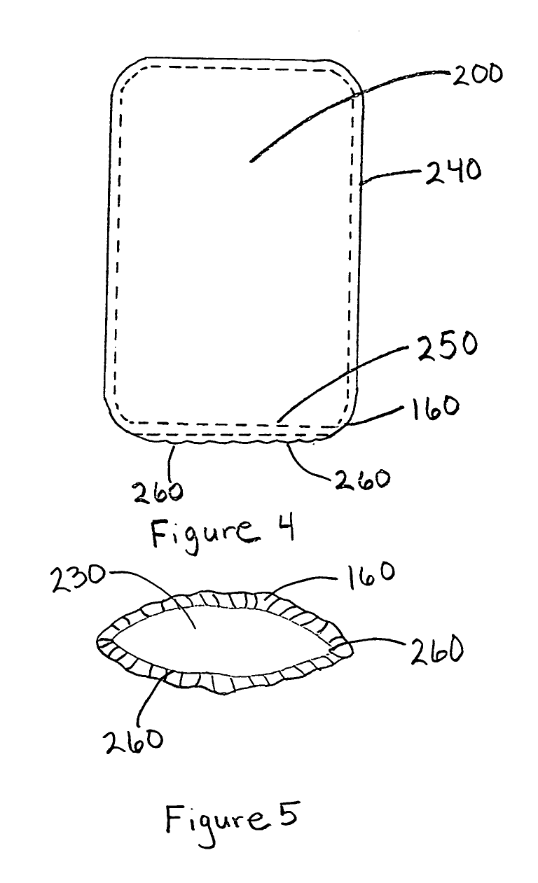

Each of the layers is disconnected from the other along one of short sides 150b and 150d, which are both formed with marginal hems. Each hem is sewn or glued separately to the elastic band 160 which a fold was formed 170 along a line of stitching 250 and 260 as seen in FIGS. 4, 5 and 6 after end pieces 160a and 160b of the elastic band is sewn or glued permanently connected.

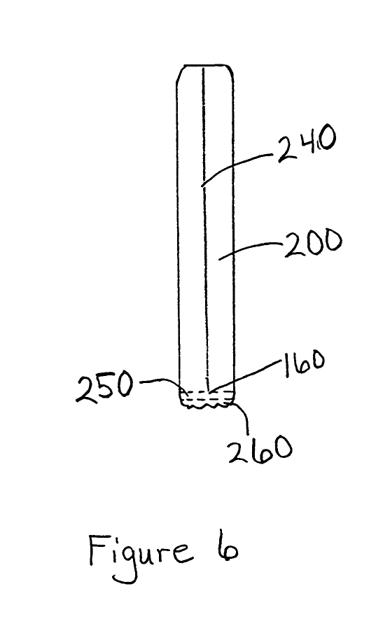

FIG. 5 By this arrangement, because of the width of the empty cavity 230 and free movement of the elastic band 160 space is provided for ease of insertion of particularly alternative nutrition/intravenous bags and poles.

1. A rectangular sack assembly utilized for concealing alternative nutrition and/or intravenous fluid bags, while hanging from an intravenous pole, having two fabric, plastic, and/or paper components combined together along two adjacent long side margins, a first side margin, and a second margin uncombined.

2. A rectangular sack assembly as set forth in claim 1, comprising a said second short uncombined margin comprising a hem wherein an elastic band is connected allowing horizontal expansion, which forms a space for easy insertion of alternative nutrition/intravenous bags and top of intravenous pole.
